(19) United States
(12) Patent Application Publication
(10) Pub. No.: US 2011/0120897 A1
(43) Pub. Date: May 26, 2011

(54) DIAPER PACKAGING

(76) Inventor: Yuki Takahashi, Mima-gun (JP)

(21) Appl. No.: 12/946,087

(22) Filed: Nov. 15, 2010

(30) Foreign Application Priority Data
Nov. 24, 2009 (JP) 2009-266376

(51) Int. Cl. A61F 13/84

(52) U.S. Cl. 206/459.5; 206/494

(57) ABSTRACT

A diaper packaging comprising a container and disposable diapers packaged therein, wherein: the disposable diaper is folded back at a fold line; a first marking which represents a size of the disposable diaper or a third marking which represents a classification of an absorptive capacity of the disposable diaper is provided on an outside of the fold line; the disposable diaper are stacked so that the fold lines face in a same direction; the container has an openable part formed on a side to which the fold lines face; a second marking which represents the size of the disposable diaper or a fourth marking which represents the classification of the absorptive capacity of the disposable diaper is provided on the container; and the first marking and the second marking are indicated in a same color, or the third marking and the fourth marking are indicated in a same color.

### BACKGROUND OF THE INVENTION

This application claims priority under 35 U.S.C. §119 to Japanese Patent Application No. 2009-266376, filed on Nov. 24, 2009, which is incorporated in its entirety by reference.

### FIELD OF THE INVENTION

The present invention relates to a diaper packaging in which disposable diapers are packaged in a container.

### DESCRIPTION OF THE RELATED ART

Conventionally, there is known a pants-type disposable diaper in which characters, which represent identification information such as a size of the diaper, is indicated on a crotch part of the pants-type disposable diaper or on an outside of the folded pants-type disposable diaper, as is described in WO 2005/023160 (Patent Document 1). In this pants-type disposable diaper, the characters which represent the identification information is provided at a location that can be recognized visually from a side of a diaper stack in which a plurality of the pants-type disposable diapers are stacked.

Patent Document 1 discloses that the characters which represent the identification information is indicated on the outside of the disposable diaper, but does not mention the relation between the identification information, such as a size, provided on the disposable diaper and a container which contains the diaper stack. In general, disposable diapers are sold at stores or brought into hospitals and care facilities in a state of being packaged in a container, before use. A user or a caregiver looks at a container packaging disposable diapers, and can recognize specification information of the disposable diapers from the container. However, attention has not been paid to easy recognition of the specification information from a disposable diaper itself. In addition, modifications have not been made for easily associating a container with disposable diapers packaged in the container.

The present invention has been achieved in view of the above circumstances, and an object of the present invention is to provide a diaper packaging in which specification information of a disposable diaper packaged in a container can be easily recognized from both the container and the disposable diaper itself, and in which the container and the disposable diaper are easily interrelated each other.

### SUMMARY OF THE INVENTION

A diaper packaging of the present invention which solves the above problems comprises a container and a plurality of disposable diapers packaged in the container, wherein: each of the disposable diaper is folded back at a fold line; a first marking which represents a size of the disposable diaper is provided on an outside of the fold line; a plurality of the disposable diapers are stacked so that the fold lines of all the disposable diapers face in a same direction; the container has an openable part formed on a side to which the fold lines of the disposable diapers face; a second marking which represents the size of the disposable diaper is provided on the container; and the first marking and the second marking are indicated in a same color.

In the diaper packaging of the present invention, a third marking which represents a classification of an absorptive capacity of the disposable diaper may be provided in addition to or instead of the first marking, a fourth marking which represents the classification of the absorptive capacity of the disposable diaper may be provided in addition to or instead of the second marking, and the third marking and the fourth marking may be indicated in a same color.

In the diaper packing of the present invention, since the first marking and the second marking are indicated in the same color, or the third marking and the fourth marking are indicated in the same color, it can be recognized at a glance that the each disposable diaper contained in the container has the specification indicated on the container when the openable part of the container is opened. In addition, even after the disposable diaper is taken out from the container, the specification of the disposable diaper can be readily recognized. Therefore, a wearer or a caregiver can pick a disposable diaper of a favorable type without confusing.

The diaper is folded back at the fold line to define one side and the other side across the fold line, and the first marking or the third marking may be provided so as to extend from the fold line to the one side and/or the other side. When the one side or the other side defined across the fold line can be visually recognized in a state where the openable part of the container is opened, the first marking or the third marking of the disposable diaper contained in the container can be more readily recognized. In addition, even in the case that the disposable diaper is taken out from the container and handled individually, the first marking or the third marking of the disposable diaper can be easily recognized.

Preferably, the disposable diaper comprises an absorbent core, and the absorbent core is folded back at the fold line. In this case, the first marking and/or the third marking is provided in a region where the absorbent core overlaps. The region where the absorbent core overlaps has high rigidity, and is unlikely to distort in spite of an effect of an elastic member disposed to the disposable diaper. Therefore, when the first marking and/or the third marking is provided in the region where the absorbent core overlaps, the first marking and/or the third marking such as characters, symbols and the like are unlikely to distort, resulting in easily recognized.

The disposable diaper used in the diaper packaging of the present invention may be a pants-type disposable diaper. Thus, the disposable diaper may be formed in a pants shape, and have a front part, a back part and a crotch part connecting the front part and the back part; and the disposable diaper may be folded back at the crotch part to form a first fold line, and may be further folded back at the front part and the back part parallel to the first fold line to form a second fold line; and the first marking or the third marking may be provided on an outside of at least one of the first fold line and the second fold line.

According to the diaper packing of the present invention, the container and the disposable diaper packaged in the container are easily interrelated each other in connection with its specification. Therefore, a wearer or a caregiver can choose a disposable diaper appropriately without confusing.

### BRIEF DESCRIPTION OF THE DRAWINGS

FIG. 1 shows a perspective view of a pants-type disposable diaper, in which a first marking which represents a size of the disposable diaper is provided on an outside of a fold line formed in a crotch part of the disposable diaper.

FIGS. 2A, 2B and 2C show an example of a folding manner of the pants-type disposable diaper shown in FIG. 1 into a quadrangular shape in a planar view.

FIG. 3 shows a perspective view of the folded pants-type disposable diaper shown in FIG. 1, that is folded according to the manner shown in FIGS. 2A, 2B and 2C.

FIG. 4 shows an example of a folded pants-type disposable diaper, that is folded into a quadrangular shape in a planar view, provided with a third marking which represents a classification of an absorptive capacity of the disposable diaper.

FIG. 5 shows an example of a folded pants-type disposable diaper, that is folded into a quadrangular shape in a planar view, provided with a first marking which represents a size of the disposable diaper.

FIG. 6 shows an example of a folded pants-type disposable diaper, that is folded into a quadrangular shape in a planar view, provided with a first marking which represents a size of the disposable diaper and a third marking which represents a classification of an absorptive capacity of the disposable diaper.

FIG. 7 shows another example of a folded pants-type disposable diaper, that is folded into a quadrangular shape in a planar view, provided with a first marking which represents a size of the disposable diaper and a third marking which represents a classification of an absorptive capacity of the disposable diaper.

FIG. 8 shows an example of a diaper stack in which a plurality of disposable diapers are stacked.

FIG. 9 shows another example of a diaper stack in which a plurality of disposable diapers are stacked.

FIG. 10 shows an example of a diaper packaging in which disposable diapers are packaged in a container.

FIG. 11 shows another example of a diaper packaging in which disposable diapers are packaged in a container.

FIG. 12 shows the diaper packaging shown in the FIG. 11 in a state where an openable part of the container is opened.

### DESCRIPTION OF THE PREFERRED EMBODIMENTS

A disposable diaper used in the present invention is to be packaged in a container before use. For example, the disposable diaper is packaged in the container, and in this state, the disposable diaper is sold at stores or brought into hospitals and care facilities.

The disposable diaper has at least one fold line in a state of being packaged in the container. On an outside of the fold line, a marking which represents a size of the disposable diaper or a classification of an absorptive capacity of the disposable diaper is provided, as described below.

Examples of the disposable diaper include: an open-type disposable diaper which is provided with a pair of fastening members on left and right sides of the diaper and which is formed into a pants shape by using the fastening members in wearing; and a pants-type disposable diaper formed in a pants shape. For example, the pants-type disposable diaper has a front part, a back part and a crotch part connecting the front part and the back part, and has a waist opening and a pair of leg openings formed by joining the front part and the back part. Concerning names of the front part, the back part and the crotch part, a part applied to an abdomen side of a wearer is called the front part, a part applied to a buttocks side of the wearer is called the back part, and a part positioned between the front part and the back part and applied to a crotch of the wearer is called the crotch part, in a state of wearing the disposable diaper. The disposable diaper preferably comprises an absorbent core provided at least at the crotch part.

In one embodiment of the pants-type disposable diaper, a laminate comprising an inner sheet and an outer sheet is formed in a pants shape to define the front part, the back part and the crotch part connecting the front part and the back part, and an absorbent body in which the absorbent core is interposed between a top sheet and a back sheet is provided at the crotch part. In another embodiment of the pants-type disposable diaper, a laminate comprising a top sheet, a back sheet and the absorbent core interposed therebetween is formed in a pants shape to define the front part, the back part and the crotch part connecting the front part and the back part.

The top sheet is preferably liquid-permeable. Examples of the top sheet include, for example, a nonwoven fabric formed from hydrophilic fibers such as cellulose and rayon; and a nonwoven fabric, formed from hydrophobic fibers such as polypropylene, polyethylene, polyester, polyamide and nylon, in which the hydrophobic fibers are hydrophilized with a surfactant on the surface thereof.

The back sheet is preferably liquid-impermeable. Examples of the back sheet include, for example, a nonwoven fabric formed from hydrophobic fibers such as polypropylene, polyethylene, polyester, polyamide and nylon; and a plastic film. As the back sheet, a laminate of a nonwoven fabric and a plastic film may be also used. Here, the term “liquid-impermeable” includes the meaning of “water-repellent”.

The inner sheet is preferably hydrophilic or liquid-impermeable, and the outer sheet is preferably liquid-impermeable. A material which can be used for the top sheet may be used as the hydrophilic inner sheet. A material which can be used for the back sheet may be used as the liquid-impermeable inner sheet or the liquid-impermeable outer sheet.

The absorbent core is not particularly restricted as long as it absorbs excrement such as urine and the like. As the absorbent core, a clump of an absorbent material, which is formed into a predefined shape, may be used, or an absorbent material wrapped with a covering sheet such as paper (for example, tissue paper), a liquid-permeable nonwoven fabric and the like may be used, for example. Examples of the absorbent material contained in the absorbent core include, for example, a hydrophilic fiber such as a crushed pulp fiber and a cellulose fiber; and an absorbent polymer such as polyacrylic absorbent polymer, cellulosic absorbent polymer, and stark-acrylonitrile absorbent polymer. The absorbent material preferably contains at least an absorbent polymer. Further, the absorbent material may be obtained by mixing an absorbent polymer with a hydrophilic fiber assembly, or dispersing an absorbent polymer on a hydrophilic fiber assembly. A shape of the absorbent core is not limited. Examples of the shape of the absorbent core include a rectangular shape, an hourglass shape, a center nipped-in gourd shape, a battledore shape and the like.

The disposable diaper preferably comprises rising flaps disposed along edges of opposite sides of the absorbent core. The rising flaps enable to prevent lateral leakage of urine and the like. The rising flap is preferably liquid-impermeable. Further, a rising elastic member is preferably provided at a free end of the rising flap in order that the rising flap rises easily.

The disposable diaper preferably comprises a waist elastic member disposed to the front part and the back part along an edge of the waist opening. The waist elastic member prevents excrement such as urine and the like from leaking from a back side or an abdomen side, even when a wearer lies. The waist elastic member may consist of a plurality of elastic members.

The disposable diaper preferably comprises a leg elastic member disposed along an edge of the leg opening. The leg elastic member prevents excrement such as urine and the like from leaking from the edge of the leg opening. The leg elastic member may consist of a plurality of elastic members.

The disposable diaper preferably comprises a plurality of body elastic members disposed to the front part and/or the back part so as to extend in a width direction of the disposable diaper. The body elastic members improve a fitting property of the disposable diaper around an abdomen region. Especially, the pants-type disposable diaper is preferably provided with the body elastic members.

Elastic materials such as a polyurethane thread, a polyurethane film, a natural rubber and the like, which are generally used for disposable diapers, can be used for the respective elastic members. The respective elastic members are preferably fixed in a stretched state with a hot-melt adhesive. For example, a polyurethane thread having a fineness of 100 dtex to 2,500 dtex is stretched at a ratio of 1.1 to 5.0 times to be fixed. A preferable hot-melt adhesive is a rubber hot-melt adhesive.

The disposable diaper is folded back at a fold line, and a marking which represents specification information of the disposable diaper is provided on an outside of the fold line. The disposable diaper is folded back at the fold line to define one side and the other side across the fold line. The one side and the other side are superposed substantially parallel each other. The marking which represents the specification information may be provided only on the outside of the fold line, or may be provided so as to extend from the fold line to the one side and/or the other side.

In the case that the marking which represents the specification information is provided on the outside of the fold line, the specification information of each of the disposable diaper can be visually recognized in a state where a plurality of the disposable diapers are stacked, as described below. In the case that the marking which represents the specification information is provided so as to extend from the fold line to one side and/or the other side defined across the fold line, the specification information of the disposable diaper can be easily recognized even when the disposable diaper is taken out from the container and handled individually.

A location where the fold line of the disposable diaper is formed is not particularly restricted; however, the disposable diaper is preferably folded back at a fold line so as to be able to be packaged in the container in a suitable manner. For example, the disposable diaper may be folded in two or three, or may be folded into a quadrangular shape (preferably, a rectangular shape) in a planar view so as to be able to be packaged suitably in a substantially quadratic prism-shaped container.

The marking, which represents the specification information of the disposable diaper and which is provided on the outside of the fold line, includes a first marking which represents a size of the disposable diaper and/or a third marking which represents a classification of an absorptive capacity of the disposable diaper. As the first marking, characters such as “S”, “M”, or “L”, which represents the size of the disposable diaper, may be indicated, or a symbol, design, or figure corresponding to the size of the disposable diaper may be indicated. As the third marking, letters such as “for 2 times” or “for 4 times”, which represent a number of urination which can be held in the diaper as the absorptive capacity of the diaper, may be indicated, or a symbol, design, or figure corresponding to the classification of the absorptive capacity may be indicated.

The first marking and/or the third marking are indicated in a specific color. Preferably, the first marking and/or the third marking are indicated in a specific color other than white. Since a general disposable diaper has a white ground, when the first marking and/or the third marking are indicated in a color other than white, the first marking and/or the third marking can be easily recognized.

Hereinafter, an embodiment for: a location where the fold line is formed; and indication of the first marking and the third marking, is described below with a pants-type disposable diaper as an example, referring to the drawings.

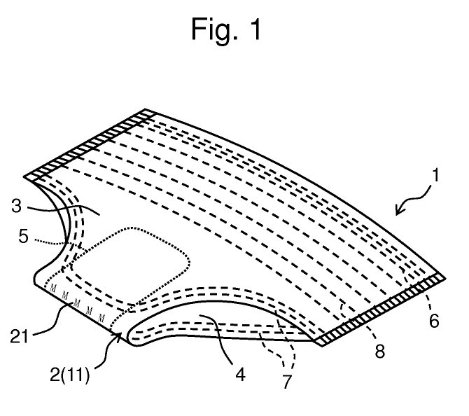

FIG. 1 shows a perspective view of a pants-type disposable diaper. In FIG. 1, the disposable diaper 1 is formed in a pants shape, and has a front part 3, a back part 4 and a crotch part 2 connecting the front part 3 and the back part 4. The disposable diaper 1 comprises an absorbent core 5 disposed so as to extend from the crotch part 2 to the front part 3 and the back part 4.

The disposable diaper 1 is folded back at a first fold line 11 at the crotch part 2. The front part 3 and the back part 4 are formed across a first fold line 11 and superposed on each other. The superposed front part 3 and back part 4 are joined to each other at their side edges to form a waist opening and a pair of leg openings. In the disposable diaper 1, a waist elastic member 6 is disposed along an edge of the waist opening, leg elastic members 7 are disposed along edges of the leg openings, and body elastic members 8 are disposed to the front part 3 and the back part 4 so as to extend in a width direction of the disposable diaper. A first marking 21 which represents a size of the disposable diaper 1 is provided on an outside of the first fold line 11. In FIG. 1, characters “M” are indicated as the first marking.

A plurality of the pants-type disposable diapers 1, each of which is in a pants shape in a planar view as shown in FIG. 1, may be stacked so that the front part 3 of the disposable diaper 1 and the back part 4 of the adjacent disposable diaper 1 are superposed on each other to form a diaper stack, and the diaper stack may be packaged in a container. However, the diaper stack is preferably formed smaller in size in order to improve a packaging efficiency. Therefore, preferably, the pants-type disposable diapers are folded into a quadrangular shape in a planar view, and the disposable diapers folded thus are stacked to form a diaper stack.

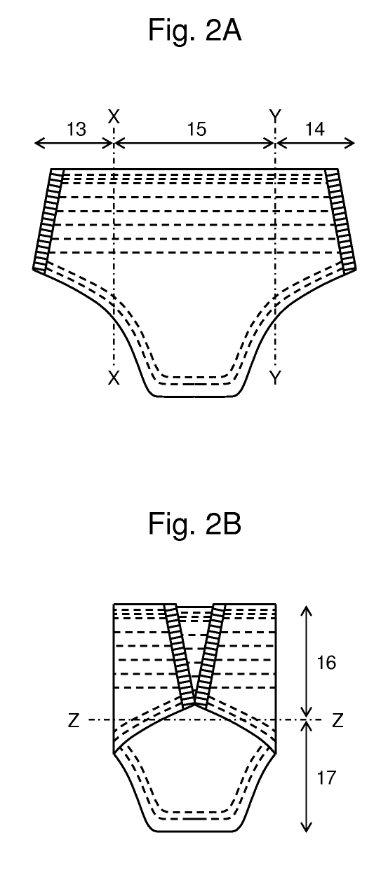

FIGS. 2A, 2B and 2C show an example of a manner of folding the pants-type disposable diaper shown in FIG. 1 into a quadrangular shape in a planar view. The disposable diaper 1 is sectioned in a width direction into a right part 13, a left part 14 and a middle part 15 therebetween, and is also sectioned in an up-down direction into an upper part 16 and a lower part 17. Here, the width direction and the up-down direction mean a right-left direction and an up-down direction, respectively, in a state where a wearer of the disposable diaper stands. The left part and the right part mean a part located on the left side of the wearer and a part located on the right side of the wearer, respectively, when the wearer wears the disposable diaper. In FIG. 2A, the right part 13 and the middle part 15 are sectioned at a line X-X, and the left part 14, the middle part 15 are sectioned at a line Y-Y. In FIG. 2B, the upper part 16 and the lower part 17 are sectioned at a line Z-Z. In the disposable diaper 1, the right part 13 is folded at the line X-X over the middle part 15 and the left part 14 is folded at the line Y-Y over the middle part 15, whereby the right part 13 and the left part 14 are superposed on the middle part 15, as shown in FIGS. 2A and 2B. Subsequently, in the disposable diaper 1, the lower part 17 is folded at the line Z-Z over the upper part 16, whereby the lower part 17 is superposed on the upper part 16, as shown in FIGS. 2B and 2C.

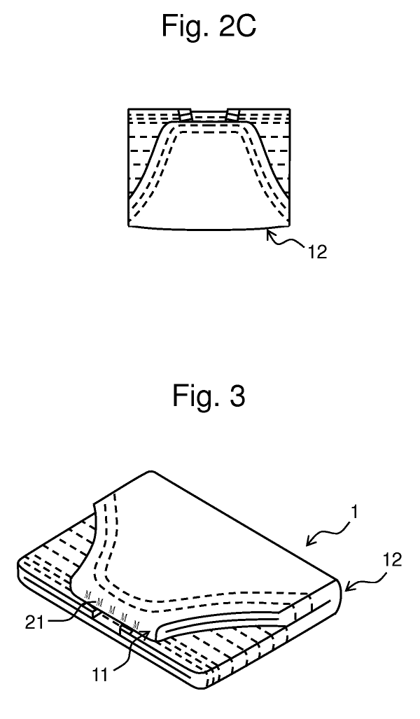

FIG. 3 shows a perspective view of the folded pants-type disposable diaper shown in FIG. 1, which is folded according to the manner shown in FIGS. 2A, 2B and 2C. In FIG. 3, the disposable diaper 1 is folded into a quadrangular shape in a planar view. When the disposable diaper 1 is folded in the manner as shown in FIG. 3, a plurality of the disposable diapers 1 are stacked to be easily put into a container. In addition, a diaper stack formed by stacking a plurality of the disposable diapers 1 becomes small in size, resulting in improving the packaging efficiency. Moreover, when the disposable diaper 1 is folded in the manner as shown in FIG. 3, the marking which represents the specification information, that is the first marking 21 in FIG. 3, and which is provided at the first fold line 11 formed in the crotch part, can be visually recognized from a side of the diaper stack, even in a state where the disposable diapers 1 are stacked to form the diaper stack.

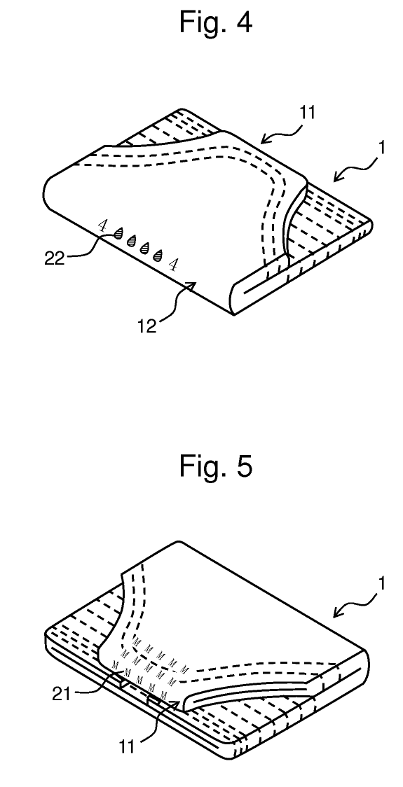

The marking which represents the specification information of the disposable diaper 1 may be provided at a fold line which is formed by folding the pants-type disposable diaper shown in FIG. 1. This is explained as follows, referring to FIG. 4.

In FIG. 4, a third marking 22, which represents a classification of an absorptive capacity of the disposable diaper 1, is provided on an outside of a fold line 12 (hereinafter, may be referred to as “a second fold line”) which is formed by folding the disposable diaper 1 at the line Z-Z in FIG. 2B. In FIG. 4, drop-shaped symbols and numerals are indicated as the third marking 22. In FIG. 4, for representing that the diaper has an absorptive capacity for 4 times of urination, four drop-shaped symbols are provided in a line together with numerals “4”. The second fold line 12 is preferably formed so as to be parallel to the first fold line 11, whereby the disposable diaper 1 is easily compact-folded into a quadrangular shape in a planar view. In this case, the second fold line 12 is formed at the front part and the back part so as to be parallel to the first fold line 11, and the marking represents the specification information of the disposable diaper is provided on the outside of the second fold line 12.

In FIGS. 1, 3 and 4, the marking, which represents the specification information of the disposable diaper, is provided only on the outside of the fold line; however, the disposable diaper is folded back at the fold line to define one side and the other side across the fold line, and the marking which represents the specification information may be provided so as to extend from the fold line to the one side and/or the other side. This is explained as follows, referring to FIGS. 5 to 7.

In FIG. 5, characters “M” are provided as the first marking 21 at the first fold line 11 formed in the crotch part, and characters “M” of the first marking 21 are also provided so as to extend to one side from the first fold line 11. In FIG. 5, the one side and the other side defined across the first fold line 11 correspond to the front part 3 and the back part 4 of the disposable diaper.

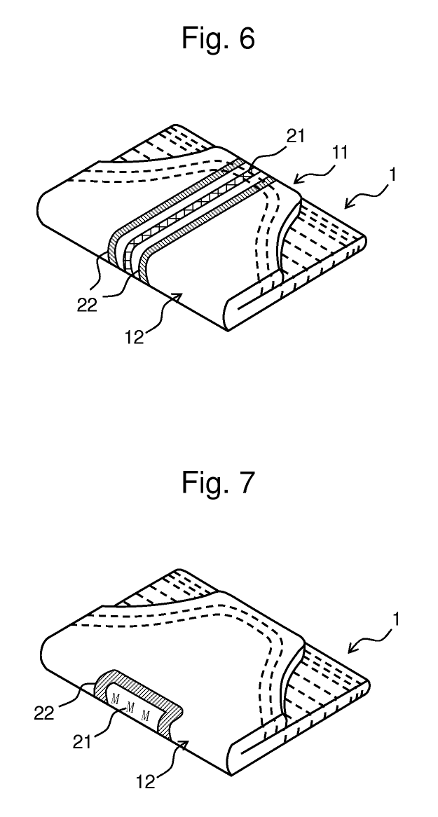

In FIGS. 1 and 3 to 5, the first marking 21 and the third marking 22 are indicated by characters, numerals, or symbols. Here, these characters, numerals, and symbols are indicated in specific colors, as described below. In addition, the first marking 21 and the third marking 22 may be indicated by a figure or design, as shown in FIGS. 6 and 7.

In FIG. 6, three straight lines are depicted so as to extend parallel to each other on the outside of the first fold line 11, on one side and the other side defined across the first fold line 11, on the outside of the second fold line 12, and on one side and the other side defined across the second fold line 12. In FIG. 6, the one side to the first fold line 11 and the one side to the second fold line 12 are the same. Of the three straight lines, the central straight line corresponds to the first marking 21, and the straight lines on both sides of the central straight line correspond to the third marking 22.

In FIG. 7, characters “M” are indicated as the first marking 21 on the outside of the second fold line 12, and a quadrangular figure, which surrounds the first marking 21, is depicted as the third marking 22. The quadrangular figure of the third marking 22 is provided so as to extend from the second fold line 12 to the one side and the other side defined across the second fold line 12.

In FIGS. 6 and 7, the first marking and the third marking are indicated in specific colors, respectively. Here, the first marking is preferably indicated in a different color from the third marking.

A method for providing the first marking and the third marking on the disposable diaper is not particularly restricted; however, the first marking and the third marking are preferably printed on the disposable diaper by inkjet printing. When printed by inkjet printing, even though the first marking and the third marking are complicated or detailed in shape or color, these markings are easily applied to the disposable diaper appropriately.

The fold line at which the first marking and/or the third marking is provided, is preferably a fold line formed by folding back the disposable diaper together with its absorbent core. In other words, the first marking and/or the third marking is preferably provided on an external surface of the disposable diaper in a region where the absorbent core overlaps. In the disposable diaper, the region where the absorbent core overlaps has high rigidity, and is unlikely to distort in spite of an effect of an elastic member disposed to the disposable diaper. Therefore, when the first marking and/or the third marking is provided in the region where the absorbent core overlaps, characters, symbols or the like of these markings are unlikely to distort, resulting in easily recognized.

A plurality of the disposable diapers are stacked to form the diaper stack. In the diaper stack, the disposable diapers are stacked so that the one side of the disposable diaper and the other side of the adjacent disposable diaper are superposed on each other. The diaper stack has both end faces in which the one side of the disposable diaper and the other side of the other disposable diaper face outside, and side faces between the both end faces, in which the fold lines or edges of the disposable diapers face outside. Here, as described above, the one side and the other side mean sides defined across the fold line at which the first marking and/or the third marking is provided. Further, in the diaper stack, a plurality of the disposable diapers are stacked so that the fold lines, at each of which the first marking and/or the third marking is provided, of all the disposable diaper face in a same direction. Therefore, in a state where the disposable diapers are stacked to form the diaper stack, the markings which represent the specification information of the respective disposable diapers can be recognized when the diaper stack is seen from the side face thereof.

In addition, when that the first marking and/or the third marking is provided so as to extend from the fold line to the one side and/or the other side across the fold line, the marking which represents the specification information of the disposable diaper can be recognized, seen from the ends face of the diaper stack.

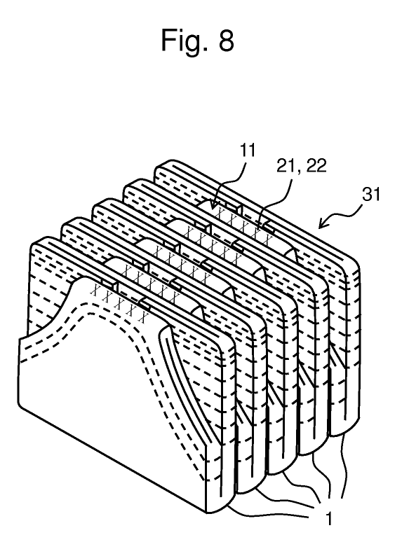

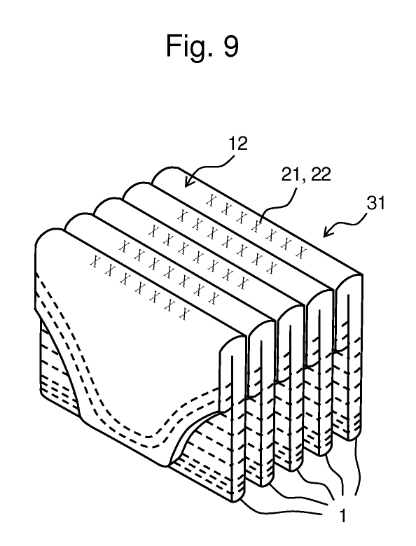

FIGS. 8 and 9 show examples of the diaper stack in which a plurality of the disposable diapers are stacked. FIG. 8 shows an example of the diaper stack in which a plurality of the disposable diapers 1, each of which is folded into a quadrangular shape in a planar view as shown in FIG. 3, are stacked. FIG. 9 shows an example of the diaper stack in which a plurality of the disposable diapers 1, each of which is folded into a quadrangular shape in a planar view as shown in FIG. 4, are stacked. In FIGS. 8 and 9, the first marking 21 and the third marking 22 are represented by “X”, for the sake of convenience.

In FIG. 8, the first marking 21 and/or the third marking 22 is provided at the first fold line 11 in the each disposable diaper 1, and a plurality of the disposable diapers 1 are stacked so that the first fold lines 11 face in a same direction to form a diaper stack 31. Therefore, the first markings 21 and/or the third markings 22 provided at the first fold lines 11 of the respective disposable diapers 1 can be recognized in a state where the disposable diapers 1 are stacked.

In FIG. 9, the first marking 21 and/or the third marking 22 is provided at the second fold line 12 in the each disposable diaper 1, and a plurality of the disposable diapers 1 are stacked so that the second fold lines 12 face in a same direction to form a diaper stack 31. Therefore, the first markings 21 and/or the third markings 22 provided at the second fold lines 12 of the respective disposable diapers 1 can be recognized in a state where the disposable diapers 1 are stacked.

A plurality of the stacked disposable diapers are packaged in a container to form a diaper packaging. The container is not particularly limited, as long as the container can contain disposable diapers, and examples of the container include a plastic film bag, a paper bag, a plastic hard container, and a corrugated fiberboard box.

The container has an openable part. In the diaper packaging of the present invention, when the openable part of the container is opened, the disposable diaper contained in the container can be taken out. Examples of the openable part include a perforation, a cover, a flap, and an adhesive tape which seals an opening of the container.

The openable part of the container is formed on a side to which the fold lines, at which the markings which represent the specification information is provided, of the stacked disposable diapers face. In other words, in the present invention, the first marking and/or the third marking is provided on at least one side face of the diaper stack, in which the disposable diapers are stacked, and the openable part of the container is formed on a side to which the one side face of the diaper stack faces. Therefore, when the openable part of the container is opened, the first marking and/or the third marking of the disposable diaper contained in the container can be readily recognized visually.

Further, if the openable part of the container is also formed on a side to which the one side or the other side diaper defined across the fold line of the disposable faces, and the first marking and/or the third marking is also provided on the one side and/or the other side defined across the fold line, the first marking and/or the third marking of the disposable diaper contained in the container can be more readily recognized when the openable part of the container is opened.

On the container, a second marking which represents the size of the disposable diaper packaged in the container and/or a fourth marking which represents the classification of the absorptive capacity of the disposable diaper packaged in the container is provided. As the second marking, characters such as “S”, “M”, or “L”, which represents the size of the disposable diaper, may be indicated, or a symbol, design, or figure corresponding to the size of the disposable diaper may be indicated. As the fourth marking, letters such as “for 2 times” or “for 4 times”, which represent a number of urination which can be held in the diaper as the absorptive capacity of the diaper, may be indicated, or a symbol, design, or figure corresponding to the classification of the absorptive capacity, may be indicated.

In first embodiment of the diaper packaging of the present invention, the first marking provided at the fold line of the disposable diaper and the second marking provided on the container are indicated in a same color. The first marking and the second marking which represent the size of the disposable diaper are preferably indicated in a different color in accordance with the size.

In the second embodiment of the diaper packaging of the present invention, the third marking provided at the fold line of the disposable diaper and the fourth marking provided on the container are indicated in a same color. The third marking and the fourth marking which represent the classification of the absorptive capacity of the disposable diaper are preferably indicated in a different color in accordance with the classification of an absorptive capacity. The third marking and the fourth marking may be changed in color in accordance with a type of the absorbent core in connection with the absorptive capacity. For example, depending on kinds or blending proportion of an absorbent material to be used in the absorbent core, the absorbent core may be changed in thickness even though having the same absorptive capacity. In such a case, the third marking and the fourth marking may be changed in color in accordance with the type of the absorbent core.

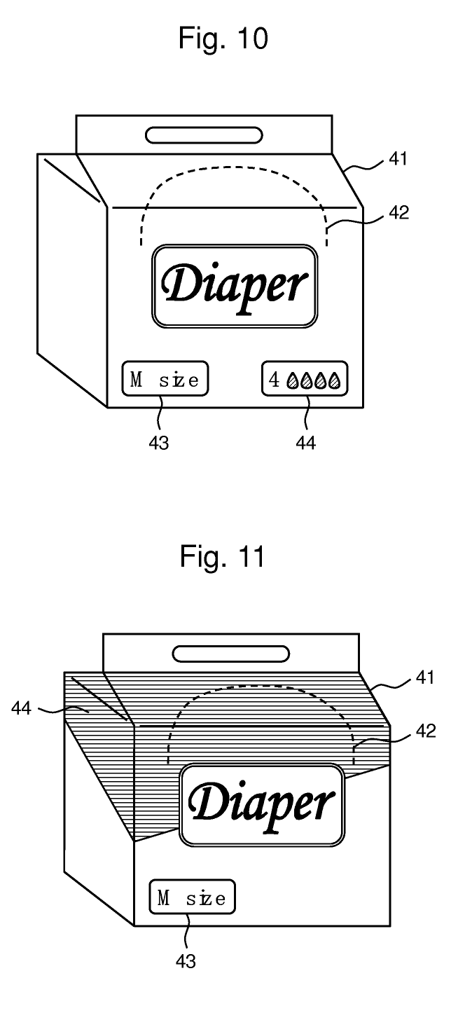

FIGS. 10 and 11 show examples of the diaper packaging in which the disposable diapers are packaged in the container. In FIGS. 10 and 11, on a container 41, characters “Diaper” are indicated as a logo for the trade name of the disposable diaper 1, and the size of the disposable diaper packaged in the container 41 or a design is also indicated. In an upper part of the container 41, a perforation is formed as an openable part 42. In FIGS. 10 and 11, the perforation is formed so as to extend from an upper surface of the container to its adjacent surface.

In FIG. 10, on the container 41, letters “M size” are indicated as the second marking 43, and four drop-shaped symbols are provided in a line together with numerals “4” as the fourth marking 44. The letters “M size” of the second marking 43 are indicated in the same color as the first marking 21 of the disposable diaper 1 packaged in the container 41, and the numeral “4” and the drop-shaped symbols of the fourth marking 44 are indicated in the same color as the third marking 22 of the disposable diaper 1 packaged in the container 41.

In FIG. 11, on the container 41, letters “M size” are indicated as the second marking 43, and a design having a specific color is applied as the fourth marking 44 to an upper half of the container 41. The letters “M size” of the second marking 43 are indicated in the same color as the first marking 21 of the disposable diaper 1 packaged in the container 41, and the design of the fourth marking 44 are indicated in the same color as the third marking 22 of the disposable diaper 1 packaged in the container 41.

For the disposable diaper 1, a set of product series may be prepared in accordance with the classifications of their absorptive capacities. Specifically, product series such as a large absorptive capacity series, a medium absorptive capacity series, and a small absorptive capacity thin type series may be prepared. In such a case, when the fourth marking 44 is provided by the design as shown in FIG. 11, it can be readily recognized the series of the disposable diaper 1 by looking at the container 41, and it is convenient. Further, since the third marking 22 is provided on the disposable diaper 1 in the same color as the fourth marking 44, the series of the disposable diaper 1 can be readily recognized also from the disposable diaper 1.

In FIGS. 10 and 11, both the second marking 43 and the fourth marking 44 are provided on the container 41; however, even in such a case, both the first marking 21 and the third marking 22 may not be necessarily provided on the disposable diaper 1 packaged in the container 41. It is only necessary that at least one of the first marking 21 and the third marking 22 is provided on the disposable diaper 1. On the other hand, in the case that both the first marking 21 and the third marking 22 are provided on the disposable diaper 1, it is only necessary that at least one of the second marking 43 and the fourth marking 44 is provided on the container 41 packaging the disposable diaper 1.

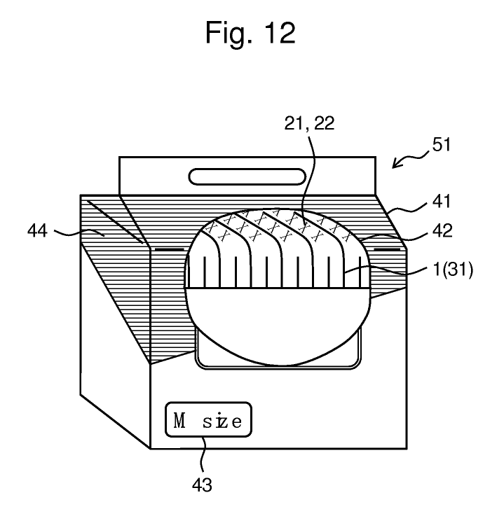

FIG. 12 shows a diaper packaging 51 of the present invention, in which the openable part 42 of the container 41 shown in FIG. 11 is opened. In FIG. 12, the openable part 42 is formed in the upper surface of the container 41, and the stacked disposable diapers 1 are packaged in the container 41 such that the fold lines, at which the first markings 21 and/or the third markings 22 are provided, of the disposable diapers 1 face upward. Thus, the openable part 42 is formed in the upper surface of the contained 41 to which the fold lines, at which the first markings 21 and/or the third markings 22 are provided, of the stacked disposable diapers 1 face. In FIG. 12, two diaper stacks 31, in each of which a plurality of the disposable diapers 1 are stacked, are stacked one above the other and packaged in the container 41. However, the number of the diaper stacks 31 packaged in the container 41 may be only one or more than two.

In the diaper packaging of the present invention, the openable part of the container is formed on the side to which the fold lines, at which the markings which represent the specification information of the disposable diapers are provided, of the stacked disposable diapers face; and further, the first marking and the second marking are indicated in the same color, or the third marking and the fourth marking are indicated in the same color. Therefore, when the openable part of the container is opened, it can be recognized at a glance that each disposable diaper contained in the container has the specification indicated on the container. In addition, even after the disposable diaper is taken out from the container, the specification of the disposable diaper can be readily recognized. Therefore, a wearer or a caregiver can pick a disposable diaper of a favorable type without confusing. In particular, at hospitals and facilities where various types of disposable diapers are always stored, the specification of the disposable diaper and the specification of the disposable diaper contained in the container can be recognized at a glance, and it is convenient.

Further, as described above, for the disposable diaper, a set of product series may be prepared in accordance with classifications of their absorptive capacities. Regarding a general disposable diaper, it is difficult to recognize the series of the disposable diaper from the diaper itself in a state where the disposable diaper is taken out from the container, even if its series can be readily recognized from the container. Meanwhile, when the third marking is provided on the disposable diaper as in the present invention, it can be readily recognized which series the disposable diaper is, even after the disposable diaper is taken out from the container.

In care facilities such as nursing homes for the aged, in particular, the diaper packaging of the present invention is suitably used. In care facilities such as nursing homes for the aged, an elderly person who may have difficulty in recognizing small characters or symbols, uses a disposable diaper. Thus, when a marking such as characters and symbols without color information is provided on a disposable diaper or a container, it tends to be difficult for such an elderly person to recognize the specification of the disposable diaper for his or her using. However, in the present invention, since the marking which represents the specification information of the disposable diaper is indicated by color, a wearer can readily recognize the specification of the disposable diaper for his or her using. In addition, comparing the first marking and/or the third marking on the disposable diaper to the second marking and/or the fourth marking on the container, an elderly person can easily choose a disposable diaper for oneself. Further, a caregiver can also recognize at a glance whether or not a wearer wears a disposable diaper of an appropriate specification, and therefore, it is convenient. Thus, the present invention is suitably applicable to disposable diapers for adults, in particular.

1. A diaper packaging comprising a container and a plurality of disposable diapers packaged in the container, wherein:
each of the disposable diaper is folded back at a fold line;
a first marking which represents a size of the disposable diaper is provided on an outside of the fold line;
a plurality of the disposable diapers are stacked so that the fold lines of all the disposable diapers face in a same direction;
the container has an openable part formed on a side to which the fold lines of the disposable diapers face;
a second marking which represents the size of the disposable diaper is provided on the container; and
the first marking and the second marking are indicated in a same color.

2. A diaper packaging comprising a container and a plurality of disposable diapers packaged in the container, wherein:
each of the disposable diaper is folded back at a fold line;
a third marking which represents a classification of an absorptive capacity of the disposable diaper is provided on an outside of the fold line;
a plurality of the disposable diapers are stacked so that the fold lines of all the disposable diapers face in a same direction;
the container has an openable part formed on a side to which the fold lines of the disposable diapers face;
a fourth marking which represents the classification of the absorptive capacity of the disposable diaper is provided on the container; and
the third marking and the fourth marking are indicated in a same color.

3. The diaper packaging according to claim 1, wherein
the disposable diaper is folded back at the fold line to define one side and the other side across the fold line, and
the first marking is provided so as to extend from the fold line to the one side and/or the other side.

4. The diaper packaging according to claim 1, wherein
the disposable diaper comprises an absorbent core, and
the absorbent core is folded back at the fold line.

5. The diaper packaging according to claim 1, wherein
the disposable diaper is formed in a pants shape, and has a front part, a back part and a crotch part connecting the front part and the back part,
the disposable diaper is folded back at the crotch part to form a first fold line, and is further folded back at the front part and the back part parallel to the first fold line to form a second fold line,
the first marking is provided on an outside of at least one of the first fold line and the second fold line.

6. The diaper packaging according to claim 2, wherein
the disposable diaper is folded back at the fold line to define one side and the other side across the fold line, and
the third marking is provided so as to extend from the fold line to the one side and/or the other side.

7. The diaper packaging according to claim 2, wherein
the disposable diaper comprises an absorbent core, and
the absorbent core is folded back at the fold line.

8. The diaper packaging according to claim 2, wherein
the disposable diaper is formed in a pants shape, and has a front part, a back part and a crotch part connecting the front part and the back part,
the disposable diaper is folded back at the crotch part to form a first fold line, and is further folded back at the front part and the back part parallel to the first fold line to form a second fold line,
the third marking is provided on an outside of at least one of the first fold line and the second fold line.
